(19) United States
(12) Patent Application Publication
(10) Pub. No.: US 2026/0301876 A1
(43) Pub. Date: Oct. 1, 2026

(54) SOFTWARE-BASED SYSTEMS AND METHODS FOR DESIGNING AND IMPLEMENTING HIGH-ORDER GATE LAYOUTS FOR SYNTHETIC LOGIC CIRCUITS

(71) Applicant: BOARD OF TRUSTEES OF MICHIGAN STATE UNIVERSITY, EAST LANSING, MI (US)

(72) Inventors: Federica Brandizzi, Okemos, MI (US); Pengfei Cao, Okemos, MI (US)

(21) Appl. No.: 19/097,754

(22) Filed: Apr. 1, 2025

Related U.S. Application Data
(60) Provisional application No. 63/572,901, filed on Apr. 1, 2024.

(51) Int. Cl. G16B 50/30; G16B 35/00

(52) U.S. Cl. CPC G16B 50/30; G16B 35/00

(57) ABSTRACT

Methods and systems for designing synthetic logic circuits to be implemented within a given host are disclosed.

### CROSS-REFERENCE TO RELATED APPLICATION(S)

This application claims the benefit of priority to U.S. Provisional Patent Application No. 63/572,901, filed on Apr. 1, 2024, the contents of each are incorporated by reference in their entireties

### STATEMENT REGARDING FEDERALLY SPONSORED RESEARCH

This invention was made with government support under DE-SC0018409 awarded by the Department of Energy. The government has certain rights in the invention.

### TECHNICAL FIELD

Various embodiments and implementations described herein relate generally to systems and methods for designing high-order, synthetic logic circuits. More specifically, embodiments and implementations hereof may involve use of a software application that designs a logic circuit to accomplish specific, user-desired outputs.

### BACKGROUND

While certain aspects of conventional technologies have been discussed herein and in the attached appendices to facilitate disclosure of the invention, such discussions in no way disclaim these technical aspects, and it is contemplated that embodiments of the present disclosure may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, any given claimed embodiment should not necessarily be construed as limited to addressing any of the particular problems or deficiencies discussed herein.

### SUMMARY

The following presents a simplified summary of one or more aspects of the present disclosure, to provide a basic understanding of such aspects. This summary is not an extensive overview of all contemplated features of the disclosure and is intended neither to identify key or critical elements of all aspects of the disclosure nor to delineate the scope of any or all aspects of the disclosure. Its purpose includes presenting some concepts of one or more aspects of the disclosure in a simplified form as a prelude to the more detailed description that is presented later.

These and other aspects of the disclosure will become more fully understood upon a review of the drawings and the detailed description, which follows. Other aspects, features, and embodiments of the present disclosure will become apparent to those skilled in the art, upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments it should be understood that such example embodiments can be implemented in various devices, systems, and methods.

In one aspect, systems and methods for generating an application-specific, modular circuit vector to implement a set of logic functions is provided. The method may include: receiving user input comprising data indicative of: a crop variety of interest, one or more desired traits, one or more undesired traits, and one or more genes of interest; accessing a library of gene information pertaining to the crop variety of interest, to obtain gene activation information relating to the one or more desired traits, one or more undesired traits, and one or more genes of interest; using the gene activation information, generating a circuit layout comprising a plurality of logic gates in which an input channel to the circuit layout corresponds to an input that affects the one or more genes of interest to affect the one or more desired traits and the one or more undesired traits; compiling a modular vector to encode the logic gates in the circuit layout for the crop variety of interest by: identifying a stored vector construct, from a database of vector constructs, for each class of logic gate used in the circuit layout; customizing the vector constructs to encode at least one logic gate input for each vector construct, the logic gate input associated with either: inputs identified as part of the gene activation information, or logic gate outputs of other vector constructs within the circuit layout; customizing at least one of the vector constructs to encode a logic gate output associated with the one or more genes of interest; combining the customized vector constructs to form the modular vector expressing the designed circuit layout; and outputting a file to the user comprising sequence information of a DNA vector corresponding to the modular vector.

In some embodiments, the input channel to the circuit layout is driven by the presence or absence of the input, the input including at least one of: a biomarker indicative of a disease state; a biomarker indicative of a stress state; and a protein that regulates the one or more genes of interest.

In some embodiments, the circuit layout further includes at least one output channel corresponding to the logic gate output and expressing an output that comprises an ‘on’ or ‘off’ regulation of one or more genes causing promotion of the one or more desired traits with depression of the one or more undesired traits.

In some embodiments, the database of vector constructs comprises stored vector constructs for each of the following classes of logic ages: NOT, AND, NAND, OR, NOR, XOR, XNOR, IMPLY, and NIMPLY.

In some embodiments, the vector constructs for each of the classes of logic gates use a common mechanism and are configured to modularly connect to one another for consistent, serial operation.

In some embodiments, the mechanism is operon-based transcriptional regulation. In some embodiment, the mechanism is DNA operon-based. In other embodiments, the mechanism is RNA operon-based.

In some embodiments, the modular vector includes at least three vector constructs linked in series, in which the logic gate output of two of the at least three vector constructs are inputs to logic gate inputs of other vectors.

In some embodiments, the method further includes formulating a logic function based on the activation information relating to the one or more desired traits, one or more undesired traits, and one or more genes of interest, the logic function defining: first inputs promoting gene activity of a first plurality of genes that up-regulate the one or more desired traits; second inputs promoting gene activity of a second plurality of genes that down-regulate the one or more undesired traits; and at least one conditional function that: (i) causes activation, through at least one second input, of at least one gene of the second plurality of genes that down-regulates the one or more undesired traits, while (ii) causing activation, through at least one first input, of at least one gene of the first plurality of genes that up-regulates the one or more desired traits but also corresponds with up-regulation of the one or more undesired traits.

In some embodiments, the method further includes transfecting a sample of the crop of interest to contain the modular vector.

In another aspect, a circuit is provided. The circuit may include: one or more input channels configured to sense one or more inputs; a plurality of logic gates arranged in a circuit layout having at least three layers, each logic gate having at least one logic gate input and at least one logic gate output; one or more output channels configured to activate a gene of interest; in which the logic gate input for each of the plurality of logic gates comprises at least one of: a DNA binding domain bound to a zip domain, a zip domain bound to an activation domain; and a zip domain bound to a repression domain; and in which the circuit layout implements a logic function including: a first gene of a crop variety to be activated upon sensing of the one or more inputs, and a second gene of the crop variety to be repressed in association with activation of the first gene.

In some embodiments, the one or more inputs include at least one of a disease biomarker or an environmentally-caused biological signal. In some embodiments, the first gene includes a tissue-specific promoter and corresponds to a desired trait. In some embodiments, the second gene corresponds to an undesired trait that, but for the circuit, would be activated in association with activation of the first gene.

### BRIEF DESCRIPTION OF THE DRAWINGS

FIG. 1 shows a block diagram illustrating examples of data flow among users and hardware according to various configurations described herein.

FIG. 2 shows a process flow diagram of an example method for developing a synthetic circuit vector.

FIG. 3 shows a schematic of a logic gate that utilizes transcription factors.

FIGS. 4A-4B show schematics illustrating single input gates.

FIG. 5 shows a schematic illustrating two-input gates.

FIGS. 6A-6B show schematics for basic logic gates for YES (FIG. 6A) and NOT (FIG. 6B).

FIGS. 7A-7B show expression patterns for YES gates encoded into tobacco leaves using naturally occurring CRE-DBD domains (FIG. 7A) and CRE mutants and core promoters (FIG. 7B).

FIGS. 8A-8D show schematics for logic gates. FIG. 8A is an example AND gate; FIG. 8B is an example OR gate; FIG. 8C is an example NAND gate; FIG. 8D is an example NOR gate.

FIGS. 9A-9D show expression patterns for logic gates encoded into tobacco leaves. The expression patterns match the desired ones defined in FIGS. 8A-8D.

FIGS. 10A-10C show three designs for encoding NIMPLY gates.

FIGS. 11A-11B are two designs for encoding IMPLY gates.

FIGS. 12A-12D are designs for XNOR gates (FIG. 12A; FIG. 12C) and XOR gates (FIG. 12B; FIG. 12D).

FIGS. 13A-13B are general designs for binary vectors (FIG. 13A) and modular vectors (FIG. 13B).

FIGS. 14A-14D are integrated circuits for traits stacking.

FIGS. 15A-15D are schematics (top) and results (bottom) for optimizing different methods of AND gate performance.

FIGS. 16A-16B are experimental results using the AND gate shown in FIG. 15A. Images of pACT2::RFP (middle), GFP (right), and the merged channels (left) are shown. A line scan (corresponding to the red line in the merged channel) is shown in FIG. 16B; the intensity of the two channels is plotted against position.

FIGS. 17A-17B are experimental results using the AND gate shown in FIG. 15C. The raw images (FIG. 17A) and enhanced images (FIG. 17B) are shown.

FIG. 18 shows an example plasmid for AND gate optimization with two-regulator Booleans (enhancer+repressed repressor).

### DETAILED DESCRIPTION

The detailed description set forth below, in connection with the appended drawings and the attached appendices, is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. In some instances, well-known structures and components are shown in block diagram form to avoid obscuring such concepts. Various embodiments, implementations, advantages, configurations, and examples of the present disclosure can also be found in the attached appendices with supporting data from experiments performed by the inventors.

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

As described below, various embodiments may provide for a software system that is capable of designing consistent, coherent, and higher order logic circuits that can effectuate specific activations within a host. In other respects, embodiments include specific logic circuits that are the product of such a process. Such embodiments may entail multiple inputs and/or multiple outputs, and can utilize a full panoply of logic gate types that utilize the same fundamental mechanism of action (or action mode), and, thus, are connectable and interchangeable in any manner so as to permit the design software to implement efficient implementations of logic gates in any configuration).

Systems described herein can achieve so-called ‘synthetic’ circuits embedded in a host to precisely regulate host activations in a coordinated manner so as to avoid penalties to desired outcomes that would otherwise be expected to arise due to host-specific cross-promotional activity (e.g., pleiotropy, inputs that cause multiple activations, etc.). And, by stacking multiple layers of individual logic gates to generate circuit layouts, high-order circuits can be achieved. High-order circuits can integrate multiple (e.g., >=2 or >=3) inputs, process programmed logic decisions, and then regulate multiple host activations. Such circuits can direct a host to exhibit a complex outcome through regulation of multiple individual activities, as well as stacking multiple outcomes together to enhance optimum host performance. And, with targeted circuit functions that activate/depress specific activations, spatiotemporal outcomes can also be defined.

Embodiments of such methods, systems, software, and circuits are described below.

### Example Hardware System

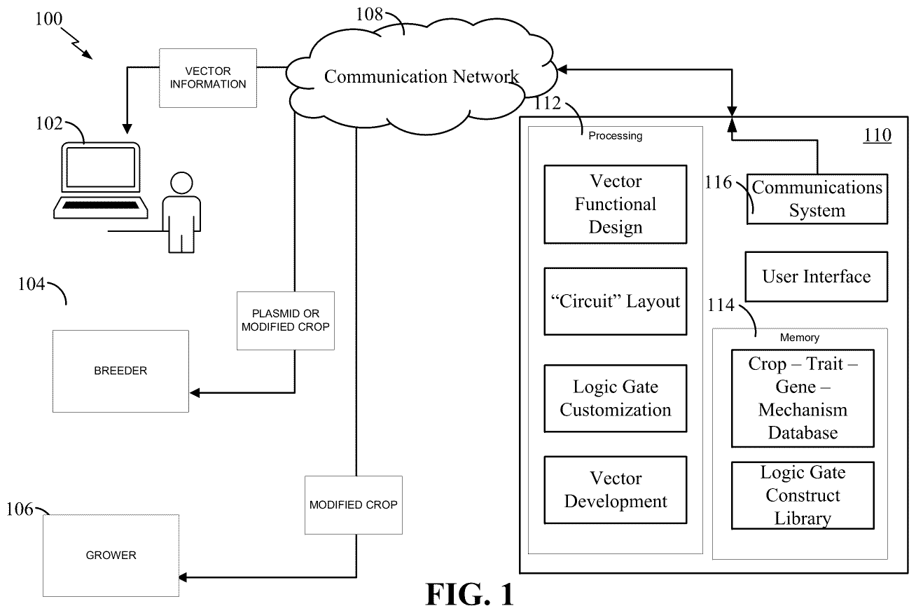

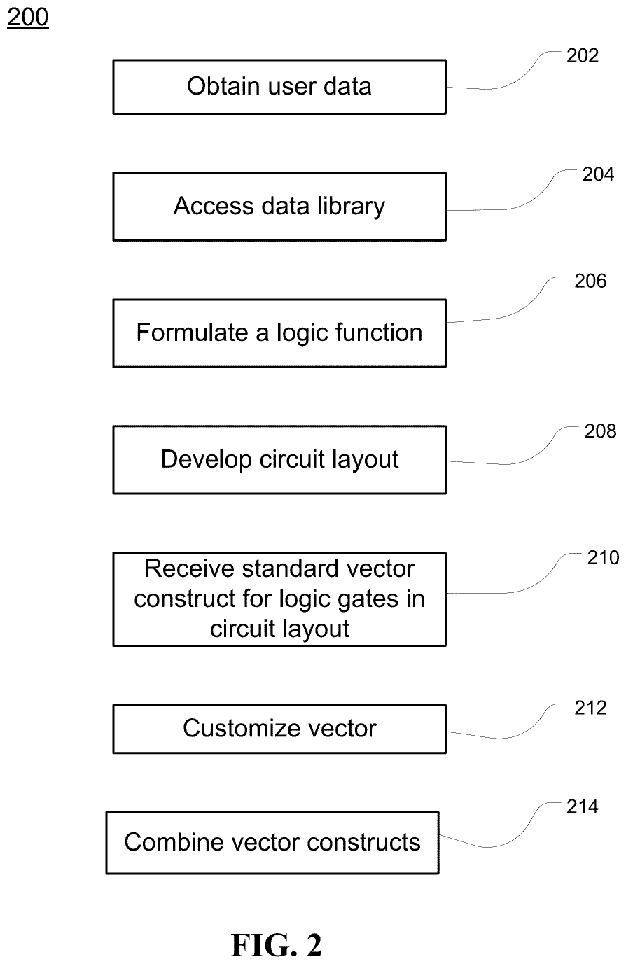

FIG. 1 shows a block diagram illustrating a system 100 for designing synthetic logic gate circuits. As shown in FIG. 1, computing device 110 can receive an input from a user (e.g., from a researcher or other user 102, a breeder 104, or a grower 106, or any other interested user, and output a variety of materials for the user derived from the result of the computing device 110 performing a process similar to that of FIG. 2.

In some examples, computing device 110 can include a processor 112. In some embodiments, the processor 112 can be any suitable hardware processor or combination of processors, such as a central processing unit (CPU), a graphics processing unit (GPU), an application specific integrated circuit (ASIC), a field-programmable gate array (FPGA), a digital signal processor (DSP), a microcontroller (MCU), etc. Processor 112 may be located within a local (to the user) device (such as a mobile device or workstation), a local area network such as a laboratory information system, may be part of a cloud-based resource, or otherwise, depending on the particular embodiment.

In further examples, computing device 110 can further include a memory 114. The memory 114 can include any suitable storage device or devices that can be used to store suitable data and instructions that can be used, for example, by the processor 112 to receive data from a user, query data from a library, generate functional designs and circuit layouts, customize vectors, etc. In some examples, a secure database may be used. The memory 114 can include any suitable volatile memory, non-volatile memory, storage, or any suitable combination thereof. For example, memory 114 can include random access memory (RAM), read-only memory (ROM), electronically-erasable programmable read-only memory (EEPROM), one or more flash drives, one or more hard disks, one or more solid state drives, one or more optical drives, cloud-based resources, etc. In some embodiments, the processor 112 can execute at least a portion of process 200, described above in connection with FIG. 2.

In further examples, computing device 110 can further include communications system 116. Communications system 116 can include any suitable hardware, firmware, and/or software for communicating information over communication network 108 and/or any other suitable communication networks. For example, communications system 116 can include one or more transceivers, one or more communication chips and/or chip sets, etc. In a more particular example, communications system 116 can include hardware, firmware and/or software that can be used to establish a Wi-Fi connection, a Bluetooth connection, a cellular connection, an Ethernet connection, a local network, an Internet connection, etc.

In further examples, computing device 110 can receive and/or transmit information (e.g., from or to a user 102, a breeder facility 104, a grower device 106, any other suitable data source, and/or any other suitable system) over a communication network 108. In some examples, the communication network 108 can be any suitable communication network or combination of communication networks. For example, the communication network 108 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

In further examples, computing device 110 can further include a display and/or one or more inputs that comprise a user interface. In some embodiments, the display can include any suitable display devices, such as a computer monitor, a touchscreen, a television, an infotainment screen, etc. to display prompts for a user to enter host and trait information and/or to display results of processes performed by the computing device 110. In further embodiments, the user interface can include any suitable input devices and/or sensors that can be used to receive user input, such as a keyboard, a mouse, a touchscreen, a microphone, etc.

### Methods and Techniques

FIG. 2 is a process flow diagram summarizing an example implementation of a method 200 for designing a circuit layout vector per various embodiments of the present disclosure. While various steps or blocks of such method 200 are specifically indicated as optional, it is to be understood that a given implementation of such method 200 need not contain all steps or blocks that are depicted (whether or not specifically identified as optional), and need not be implemented with steps or blocks in the same order as depicted. In some embodiments, process 200 may be implemented as a software program, such as a software-as-a-service.

At block 202, process 200 begins with obtaining data from a user that indicates which host of interest is to be addressed with the resulting circuit layout, as well as specific outcomes that are desired versus undesired. In some embodiments, the host of interest may be such that a given outcome of interest need not be specified by a user as it would be readily appreciated for the given host. For example, where the host is a crop variety such as rice, optimized yield (number of grains per plant, volume of grains, etc.) may be preprogrammed to be a desired outcome of interest, while undesirable traits (e.g., drought or temperature intolerance) may be preprogrammed to be undesired outcomes of interest.

In some examples, a user may be prompted to define a crop variety of interest, which may be widely recognized for having specific traits that were bred-in. Thus, the bred-in traits may automatically be set as desired traits. In further examples, a user may define both spatial information (e.g., where within the host a desired outcome should occur) and/or temporal information (the outcome should occur only when a certain circumstance is detected).

In some examples, a user may specifically identify one or more genes of the crop variety that are related to the desired or undesired traits. This information may further include both the input that activates the gene as well as the trait or traits promoted by activation of the gene.

At block 204, a data library is then accessed for the host identified by the user. The data library may contain records that correlate information pertaining to activations that are possible to be promoted or depressed for the given host. For example, for a given crop variety, the data library may contain records that correlate inputs (e.g., proteins, biomarkers, environmental stressors, disease states, etc.) with known genes activated by those inputs (whether directly or through signaling pathways), and further correlate traits promoted by those genes. In some embodiments, the data library may associate inputs with one or multiple activations (e.g., genes), may associate a single activation with multiple outcomes, and may associate a single outcome with multiple activations.

For embodiments in which the host of interest is an organism (e.g., yeast, bacteria, plant, crop, etc.), the data library may contain information regarding known genes that promote or depress specific traits. Given gene pleiotropy, a single gene may promote both a desired and undesired trait. And, similarly, a given protein or biomarker may cause activation (directly or indirectly) of multiple genes.

Thus, at block 204, the user-defined or pre-programmed outcomes or traits that are desired and undesired for a given host can be used to determine (1) which genes or activations affect the desired and undesired traits; (2) to what extent those genes or activations promote other traits; (3) which inputs activate those genes or activations; and (4) which additional genes or activations are also activated by those inputs. Based on this information, a circuit layout can be developed.

Optionally, at block 206, a logic function can be formulated based upon the user input data and upon the information queried from the data library. For example, if a user desires to activate an anti-viral function in a specific location only when virus activity is detected, but wants the anti-viral function not to negatively impact output of the host, a spatio-temporal logic function can be developed using information from the data library. In the circumstance of the host being a crop variety, the process 200 may identify a tissue-specific promotor/gene (TSPG) that activates a disease resistance attribute and identify one or more biomarkers that activate that promotor/gene (BioAct). However, it may be known that activation of that TSPG tends to depress plant growth. Therefore, a second gene or set of genes (G2) may be identified that promotes plant growth, as well as the inputs (e.g., proteins, biomarkers, etc.) that activate those genes (G2In). Thus, a logic function may be developed that states: when BioAct=true, then G2In=true. In other embodiments a state table can be developed to describe how BioAct being present or not present relates to causing production (or overexpression) of G2In.

At block 208, a circuit layout is developed to cause the desired traits and prevent the undesired traits for the host of interest, given the information from the data library and/or the logic function. In some embodiments, a known code-to-HDL program may be utilized to convert a state table or logic function into a circuit layout that will achieve the desired activations and outcomes. Software applications that may be used to automatically develop a logic gate layout include both open source and readily available programs for developing logic gate diagrams (e.g., Yosys and Logic Friday), as well as sophisticated VLSI and VHDL design suites such as those offered by Cadence Design Systems, Mentor Graphics, and Synopsys, and tools for planning FPGA (Field-Programmable Gate Array) synthesis. However, rather than being limited to a specific logic gate/transistor type (e.g., NAND or NOR gate based design), the electronic design automation software may be set to permit use of any type of logic gate in developing the circuit layout.

At block 210, the process 200 retrieves a standard or predefined vector construct for each type of logic gate involved in the circuit layout from block 208. In some embodiments, the vector constructs may come from a library of vector constructs specific to the host of interest. For instance, the examples described below as well as in U.S. Provisional Application No. 63/572,901 describe vector constructs for a variety of logic gates for crops.

At block 212, the vectors are customized so that their inputs and outputs will connect to the appropriate channel. Some vectors (acting as logic gates) may have input channels that are customized to receive the output of another vector as its input. Other vectors may be customized so that one or more of their input channels are customized to detect the presence of a given protein, biomarker, signal, etc. And, at least one vector may have as its output a given protein or other output that will drive the trait of interest.

At block 214, the customized vector constructs are then combined (in series, parallel, etc.) per the circuit layout to form a modular vector. The modular vector may be output as a sequence listing, or may be utilized to develop one or more plasmids for modification of the host/crop of interest.

### Example System Interactions

With reference to FIG. 1, examples of data interactions among resources and entities will now be described. It is to be understood, however, that any given facility, resource, user, or location depicted in FIG. 1 need not be part of a claimed system.

In one example, a user 102 may log into a portal through a communication network (e.g., Internet website) 108. A user interface may be provided to the user 102, that prompts the user to enter certain data, which as described above may include identification of a host of interest, desired traits or outcomes, undesired traits or outcomes, spatio-temporal information, and/or information on genes or other functions related to the foregoing. The user enters that information, and it is communicated to a remote server 110. The server queries a mechanism database or library (e.g., a Crop-specific Trait→Gene→Input library) to determine a set of inputs and a set of genes/functions that will promote the desired traits and depress the undesired traits.

From this information, the server 110 may develop an algorithm or logic function that reads one or more inputs and causes positive and/or negative impacts on the traits of interest. This algorithm or logic function is then used to develop a logic gate circuit layout. For each class of logic gate used in the circuit layout, the server 110 may retrieve a logic gate construct, such as a vector representing how the logic gate would be instantiated in the host of interest. Each logic gate is then customized by the processor to ensure the gates fit together per the circuit layout and ‘read’ the appropriate inputs and ‘cause’ the appropriate outputs. The processor then combines data regarding the customized vectors/gates to develop a modular vector.

An output file containing the developed vector may be the sole output sent back to the user. Alternatively, the server 110 may be part of a facility or organization that also develops a material (e.g., plasmid, RNA, etc.) that will embody the developed vector and cause it to be embedded within the host of interest. Thus, in some embodiments, the server 110 may send information regarding such a plasmid to a user such as a breeder, or may output an order to ship a physical sample of the plasmid to the breeder.

In yet further embodiments, the server 110 may output an order to a laboratory to generate the plasmid and use it to modify a sample host (e.g., sample crop or plant), so that the material sent to a breeder or grower includes a modified crop that was modify per the developed vector.

### Hardware and Software Infrastructure Examples

The present invention may be embodied on various computing platforms that perform actions responsive to software-based instructions and most particularly on touchscreen portable devices.

A computer readable medium or a memory may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any non-transitory, tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code and software instructions stored on a computer readable medium or memory may be stored and retrieved using any appropriate medium, including but not limited to wireless, wire-line, optical fiber cable, radio frequency, etc., or any suitable combination of the foregoing. Computer program code for carrying out operations for aspects of the present disclosure may be written in any combination of one or more programming languages, including an object oriented programming language such as Java, C#, C++, Visual Basic, VHDL, or the like and conventional procedural programming languages, such as the “C” programming language or similar programming languages.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices (such as through an application programming interface) to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are, accordingly, to be regarded in an illustrative sense rather than a restrictive sense.

### Benefits of Genetic Circuits

Biosynthesis-growth tradeoff is a common in critical issue in crop metabolic engineering. This is frequently complicated by gene pleiotropy, in which a single gene influences multiple, sometimes seemingly unrelated, traits. Overexpression of biosynthetic enzymes may accumulate essential amino acids, but compromises plant growth. For instance, overexpression of the enzyme CSLF6 accumulates mixed-linkage glucan (MLG) but compromises plant growth. In another example, constitutive overexpression of WRI1, a master transcription factor, accumulates fatty acids/oil, but comprises plant growth. In a more general example, plants may be modified to be resistant to a given disease, but said resistance leads to a lower yield of the plant.

The responsible genes for most valuable traits in common crops have been identified. “Responsible gene” refers to genes which “give” a plant a trait when overexpressed or deleted. However, modulating the responsible gene frequently leads to severe side effects. Therefore, a method of programmable logic regulation of gene expression is attractive, and the gene can be used as an output.

For example, one can search for key words relating to a plant or a trait of interest using literature databases such as PubMed, NCBI, or Phytozome, which produce reviews and research of individual genes, sequences of candidate genes, and side effects of modulating the genes. For some well-studied crop species, databases summarize major traits and the responsible genes. Rice Data Center (ricedata.cn/gene) presents major traits in rice. For example, rice blast (Magnaporthe grisea) s a common fungus can cause serious damage to rice. 84 genes play a role in establishing resistance to rice blast (ricedata.cn/gene/gene_pi.htm). Similarly, Maize Genetics and Genomic Database (maizegdb.org) provides a database for maize traits and phenotypes.

In many cases, expressing or repressing a single gene is effective in producing a desired trait. This gene can be chosen to be included in a circuit as an output. This is the case for most plant disease resistance and environmental resistance (drought, heat, lodging, etc.) traits. For more complicated traits that are quantitatively affected by many genes, there is no easy way to select one or a few genes as output. This is the case for plant architecture, photosynthesis, and other traits.

One possible solution to trait tradeoff is dynamic and precise gene regulation. In some instances, this can be achieved by breaking gene pleiotropy. It is of particular interest to develop methods such that a gene can be expressed or repressed as necessary only at specific times. For growth and development, genes could be modified to specific expression levels in particular tissue types and growth stages. For plant immunity, plants could express/repress defensive genes only when pathogens are present in defensive tissue. For abiotic stress, gene expression could be activated under specific conditions (e.g., temperature exceeding a particular level for a given amount of time). This could also be used to develop de novo design and implementation of artificial signaling pathways.

Additionally, dynamic and precise gene regulation can be used to achieve traits stacking by regulating multiple genes. This requires coordinating multiple genes' expressions to achieve one complex trait. For instance, achieving four desired traits could require stacking the expression of four genes corresponding to each trait. Thus, a high-order circuit can be designed to achieve multiple traits' stacking.

Gene editing refers to permanently altering a gene's expression and changes. In contrast, gene regulation refers to transiently altering a gene's expression. This allows for dynamic and precise gene regulation, specifically the ability to determine when, where, and in what condition a gene is expressed. It may also be used to determine the level of expression in each of those instances. To do so, one can take advantage of natural transcriptional regulation (e.g., natural zinc fingers or DNA binding domain (DBD)-DNA pairs), or construct next-generation synthetic transcription factors. For instance, one could take advantage of proteins with DNA-binding domains that only bind in the presence of certain enzymes or proteins.

There has been some previous, limited research into building logic gates in plants. For instance, some researchers have used bacterial repressor proteins binding to DNA elements to build AND, OR, NAND, NOR, NIMPLY, and IMPLY gates. Some researchers have used bacterial repressor proteins binding to DNA elements or DNA recombinase to build AND, OR, NAND, NOR, and NIMPLY gates. Other researchers have used DNA recombinase to build AN, OR, NOR, and NIMPLY gates.

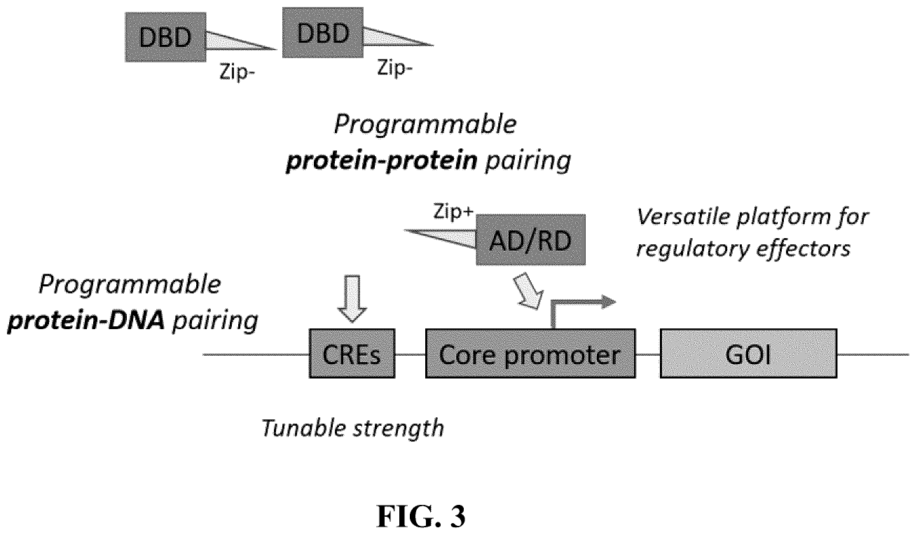

FIG. 3 is a schematic demonstrating one example of how to construct next-generation synthetic transcription factors. DNA binding domains (DBDs) are attached to Zip− domains (e.g., bZip, a zipper domain which is a specific transcription factor). Zip+ domains are bound to AD/RD regions, which can bind to promoters in a DNA sequence. Essentially, when the DBD/Zip− compounds bind with the Zip+/AD/RD compounds, the promoter will be activated and the gene of interest (GOI) will be transcribed into RNA. This method offers programmability, because a wide range of genes can be selected to regulate. It also offers single-layer regulation, which increases efficiency because minimizes expression noise and delay. This method is highly versatile and can simultaneously integrate multiple (greater than equal to two, or greater than or equal to three) inputs and multiple outputs. Additionally, using this basic method allows the creation of a toolkit of customizable binary vectors and modular vectors.

There are many potential pairs of artificial zinc fingers (ZFs) and other orthogonal DBD-DNA pairs. The DBD size is critical for efficiency of dynamic regulation. The average size of ZFs is between 7 amino acids (which targets ~3 base pairs) and 80 amino acids (which binds to ~9 base pairs). For a three-nucleotide combination, there are 64 (4×4×4) unique sequences the DBD may target.

There are many natural transcription factors (TFs) which form CC homodimer/heterodimers that may be selected as elements of this method. Additionally, there are between dozens and hundreds of bZip− derived CC homodimer/heterodimers. Should naturally occurring or bZip− derived CC homodimer/heterodimers be insufficient, there are hundreds of de novo designed CC homodimer/heterodimers. This opens up a wide array of pairs of synthetic coiled-coil homodimer/heterodimers that may be used to customize a genetic circuit.

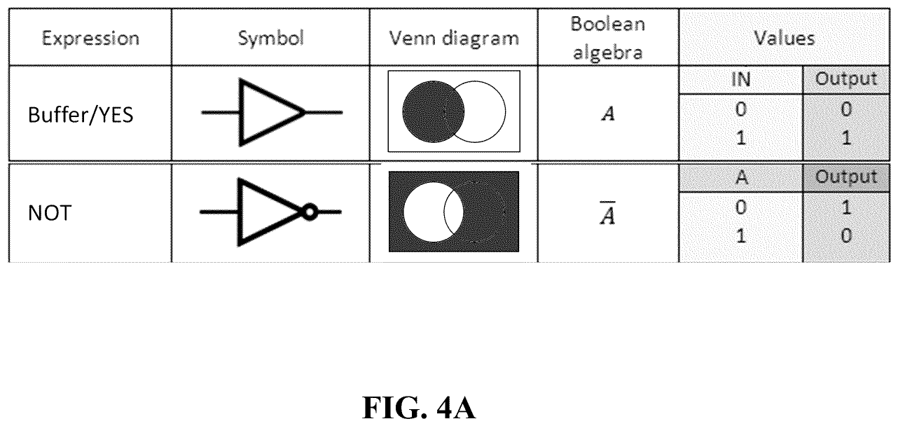

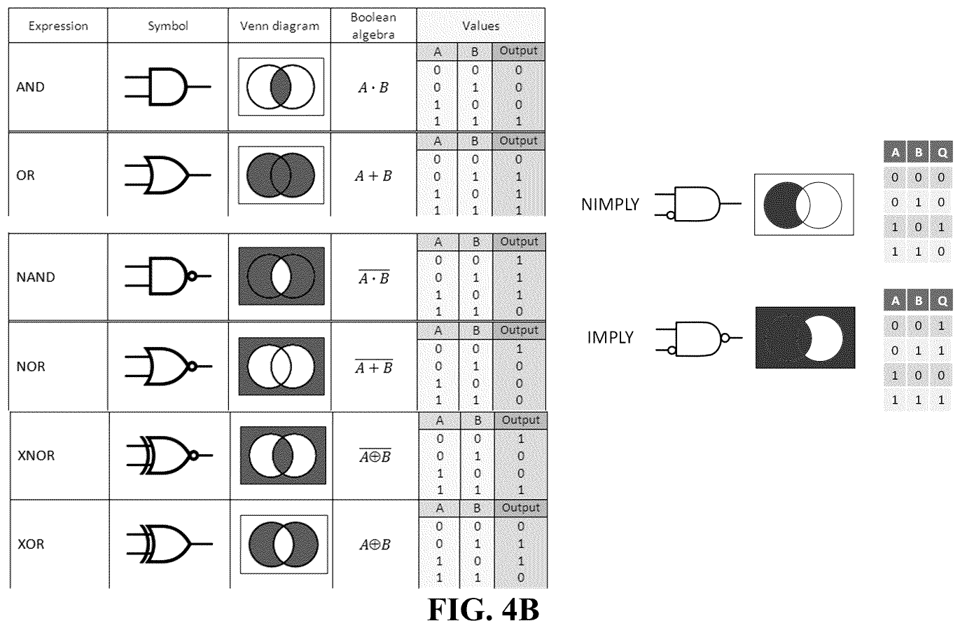

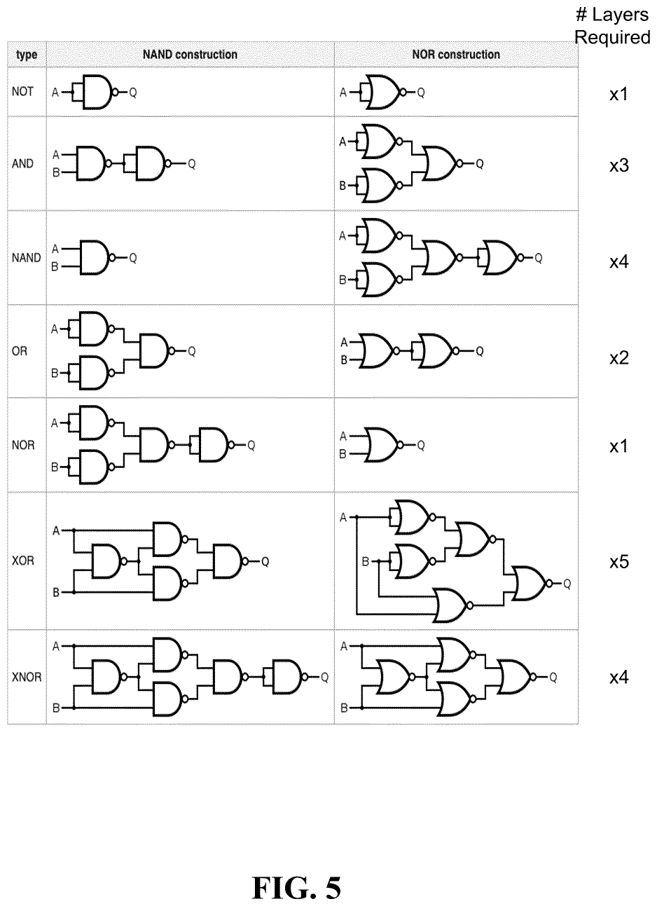

In one embodiment, logic gates are constructed in tobacco leaves. The most basic gates are single input gates (shown in FIGS. 4A-4B). In a single input gate, the expression can be “Buffer/YES” or “NOT”. The strength of regulation can be tuned for the DBD and/or for the DNA. For the DBD, the strength may be tuned by cooperative effects by TF dimerization, or mutated ZF variants with different DNA-binding affinity. For DNA, the strength may be tuned by the copy number of operators, a mutated operator, or core promoters. Other gates can be two-input gates (see FIG. 5). These include AND, OR, NAND, NOR, XNOR, and XOR. Non-basic logic types that are frequently used in computer science and synthetic biology include NIMPLY AND IMPLY.

Universal NAND and NOR gates can be layered to build all the other basic logic types. However, this strategy has increased delay, amplified noise, and limited number of bacterial repressor-operator pairs. In contrast, the method presented herein provides all basic logic functions with much higher efficiency with single-layer regulation. Each processing NOT/NOR/NAND gate is one layer; each operator-matching repressor protein (TetR, LacI, CI, etc.) is one regulator. In a given circuit, the number of layers can be less than or equal to the number of available regulators. FIG. 5 shows examples of how logic functions can be constructed, and the number of layers/regulators required for each logic function.

Importantly, the systems and methods described herein enable building all six two-input logic gates (AND, OR, NAND, NOR, XOR, and XNOR) and two additional logic types (IMPLY and NIMPLY) that are frequently used in synthetic biology. Each logic type can be materialized using various mechanisms. Previous work has been limited to only materializing a few logic types, but has fallen short of attaining all eight desired logic types in a single toolkit. Furthermore, previous work has been unable to achieve XOR and XNOR gates in logic circuits in plants.

Thus, for the first time, the systems and methods described herein are able to materialize eight logic gates, including XOR and XNOR, in a single toolbox. This is a significant advantage over previous work.

### Methods of Building a Modular Vector

Once a genetic circuit is designed, it is then possible to build the vector such that it may be expressed in a host system. A custom vector refers to a vector that encodes one or more logic gates to control gene or protein expression.

### Polynucleotides and Proteins/Peptide

The terms “polynucleotide” or “nucleic acid” are used interchangeably herein and refer to a polymeric form of nucleotides of any length, either ribonucleotides or deoxyribonucleotides. Thus, this term includes, but is not limited to, single-, double- or multi-stranded DNA or RNA, genomic DNA, DNA-RNA hybrids, or a polymer comprising purine and pyrimidine bases, or other natural, chemically or biochemically modified, non-natural, or derivatized nucleotide bases. These terms also refer to complementary DNA (cDNA), which is DNA synthesized from a single-stranded RNA (e.g., messenger RNA (mRNA) or microRNA (miRNA)) template in a reaction catalyzed by the enzyme reverse transcriptase. The backbone of the polynucleotide can comprise sugars and phosphate groups (as may typically be found in RNA or DNA) or modified or substituted sugar or phosphate groups.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “sequence identity” as used herein refers to the extent that sequences are identical on a nucleotide-by-nucleotide basis or an amino acid-by-amino acid basis over a window of comparison. Nucleic acid and protein sequence identities can be evaluated by using any method known in the art. For example, the identities can be evaluated by using the Basic Local Alignment Search Tool (“BLAST”). The BLAST programs identify homologous sequences by identifying similar segments between a query amino or nucleic acid sequence and a test sequence which is preferably obtained from protein or nucleic acid sequence database. The BLAST program can be used with the default parameters or with modified parameters provided by the user.

The term “percentage of sequence identity” is calculated by comparing two optimally aligned sequences over the window of comparison, determining the number of positions at which the identical nucleic acid base (e.g., A, T, C, G) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The term “substantial identity” of polynucleotide sequences means that a polynucleotide comprises a sequence that has at least 95% sequence identity to the polynucleotide encoding the polypeptide of interest described herein. Alternatively, percent identity can be any integer from 95% to 100%. In one embodiment, the sequence identity is at least 95%, alternatively at least 99%. More preferred embodiments include at least: 96%, 97%, 98%, 99% or 100% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

In some preferred embodiments, the term “substantial identity” of amino acid sequences for purposes of this invention means polypeptide sequence identity of at least 95%, preferably 98%, most preferably 99% or 100%. Preferred percent identity of polypeptides can be any integer from 95% to 100%. More preferred embodiments include at least 96%, 97%, 98%, 99%, or 100%.

“Regulating” expression of a gene or protein refers to controlling whether or not the gene is expressed, or under which conditions it is expressed.

“Repressing” or “depressing” a gene or protein refers to decreasing or eliminating expression of the gene or protein. This can be referred to as an “off” state of the gene.

“Expressing” of a gene or protein refers to promoting translation/transcription of the gene or protein. A gene or protein may be over expressed, which refers to a higher level of expression compared to expression in a wildtype plant (e.g., non-mutated) under regular growth conditions. Expressing may also be referred to as promoting. This can be referred to as an “on” state of the gene.

A “mechanism” may refer to a mechanism of activation for a gene to be expressed or repressed. For instance, a mechanism may be protein-protein gated, or protein-DNA gated. In some embodiments, vectors can be designed such that multiple logic gates use the same mechanism and are configured to connect to one another for consistent, serial operation. For instance, a vector may be designed such that every gene is expressed in the presence of a certain protein. A mechanism may be operon based. In other words, a gene may be activated or inhibited by acting on a DNA operator that leads to expression of said gene. A mechanism may be DNA operon-based transcription regulation. Alternatively, a mechanism may RNA operon-based.

### Biomarkers

In some embodiments, logic circuits are built to activate or deactivate based on the presence of an input. In some embodiments, an input may be a biomarker. Biomarkers include, but are not limited to, proteins, nucleic acids (e.g., sRNA), transcription factors, metabolites, volatile organic compounds, phytohormones, hormones, enzymes, and microbials.

In some embodiments a biomarker may be indicative of a state, such as a desirable or undesirable state. For instance, a biomarker may be indicative of a disease state or a stress state. “Indicative” refers to occurring primarily when the plant is in a certain state. For instance, the presence of an enzyme may be highly correlated with a disease; therefore, the presence of said enzyme is “indicative” of a disease state.

A “disease state” refers to a state in which a plant is infected with a disease (e.g., fungus, bacteria, or virus) or suspected of being infected with a disease. A disease state may refer to the negative health of a plant. For instance, in a disease state, a plant may have stunted growth, discolored leaves or lesions on leaves, wilting, deformations, or rot.

A biomarker may be environmentally caused. For instance, a biomarker may be present due to light, temperature, soil conditions, water, nitrogen, nutrients, humidity, phenolic compounds, terpenoids, or pollutants. A “stress state” refers to a state in which a plant is growing in adverse or non-ideal environmental conditions.

### Selecting Elements for a Modular Vector

In some embodiments, binary vectors for each basic logic type can be constructed for specific genes. Multiple vectors may be constructed for a single trait, which leads to a higher level of possible customization. For instance, protein-protein gated, or protein-DNA gated vectors may be used. In addition, vectors that rely on buffers for activation/repression may be used. A binary vector may be selected based on the trait the vector is associated with, the input required to activate/deactivate the vector, and the output of the vector.

A modular vector may then be built using one or more binary vectors. There may be multiple choice for any genetic part (e.g., DBDs, peptides, CREs) for fine-tuning and building integrated circuits. By combining different constructs, many modular vectors may be made.

In some embodiments, a library or database of binary vectors is stored on a computer. A user may then enter a desired trait, or a protein of interest, and retrieve binary vectors related to said desired trait. A modular vector may then be designed based on the retrieved binary vectors. If a user has multiple desired traits, multiple binary vectors may be retrieved and combined into a single modular vector.

All binary vectors were assembled using the vector backbone of pAGM4673 (see Engler, C., et al. ACS Synthetic Biologic, 3.11 (2013): 839-843). The vector backbone of pAGM4673 contains required DNA elements for binary vectors propagation in bacterium for vector construction and agrobacterium for agrobacterium-mediated plant transformation.

In some embodiments, for vector construction in bacterial E. coli, DNA elements (promoters, coding sequences, terminators, etc.) were assembled into plasmids (binary vectors). Next, the assembly plasmids (binary vectors) were transformed into Agrobacterium and the into plants using Agrobacterium-mediated methods.

In some embodiments, vectors were made in two steps. First, individual DNA elements (e.g., promoters, coding sequences, terminators, etc.) where generated, using PCR amplification from plants, long DNA synthesis by Integrated DNA Technologies and Twist Bioscience, annealing short oligos synthesized by Thermo Fisher Scientific. Second, individual DNA elements were assembled into the vector backbone of pAGM467 using the Golden Gate method (see Engler, C., et al. ACS Synthetic Biologic, 3.11 (2013): 839-843).

Performance prediction of a vector can be done using multiple approaches. Previously published papers provide fundamental predictions. For instance, some papers will report that Promoter A directs expression in plant stems, and thus Promoter A is identified as a candidate input. Additionally or alternatively, optimization can be predicted by comparing interchangeable DNA elements. For example, if both Promoter A and Promoter B have potentially similar performance, they can be individual tested in a tobacco (Nicotiana tabacum) transient expression assay. A tobacco transient expression assay allows transient expression of the plasmid in tobacco leaves using agrobacterium-mediated transformation. In some embodiments, a plasmid logic gate controls a green fluorescent protein (GFP) reporter as an output. The fluorescent intensity of GFP indicates the output intensity, which reflects the designed logic gate/circuit performance.

### Example Host Systems

Host systems may refer to a cell, a seed, or a plant cell.

As used herein, a “plant cell” may include any type of plant cell from any plant species. Suitable plants cells may include dicotyledonous plant cells or cells from broad leaf plants including, without limitation, a soybean plant cell, a common bean plant cell, or a leguminous plant cell. In some embodiments, the plant cell comprises a stem, root, or leaf cell. Plant cells also include plant callus or other plant tissues composed of plant cells. In one example, a plant of interest may be rice (Oryza sativa). Rice blast is a significant disease for rice. Constitutive overexpressing or deleting many genes can give plant resistance to rice blast, but also compromise yield, which is called growth-defense tradeoff. Therefore, logic control of gene expression that only relies upon disease strike is attractive. In another example, a plant of interest may be wheat (Triticum aestivum). Wheat stripe rust is a significant disease for wheat crops. Constitutive overexpressing or deleting many genes can give plants resistance to wheat rust but also cause severe side effects.

In another aspect of the present invention, plants are provided. The plants may include any one of the plant cells described herein. The plants may include plants in which every cell of the plant is a plant cell modified as described herein. Alternatively, the plants may include plants in which only certain tissues within the plant include the plant cells described herein.

As used herein, a “plant” includes any portion of the plant including, without limitation, a whole plant or a portion of a plant such as a part of a root, leaf, stem, seed, pod, flower, tissue plant germplasm, asexual propagate, or any progeny thereof. For example, a soybean plant refers to the whole soybean plant or portions thereof including, without limitation, the leaves, flowers, fruits, stems, roots, or otherwise. Suitable plants may include dicots or broad leaf plants including, without limitation, a soybean plant, a common bean plant, or a leguminous plant.

The plant may exhibit improved properties over a control plant. Properties may be selected by a user. As used herein, a “control plant” is a plant that has not been modified as described herein. Exemplary control plant cells may include those from a tobacco plant variety.

### Methods of Expressing a Custom Vector in a Host

Various methods may be used to express a custom vector in a host. For instance, Agrobacterium-mediated transformation may be used.

In some embodiments, expression cassettes for each logic gate were assembled using the Golden Gate method into a binary vector with modified backbone of pAGM4673 (Engler, C., et al. ACS Synthetic biologic 3.11 (2014): 839-843). The binary vector was transformed into Agrobacterium strain GV3101 and then transformed Arabidopsis using the floral dip method (Clough, S. J., and Bent, A. F. The Plant Journal 16.6 (1998): 735-743).

### Genetic Circuits Examples

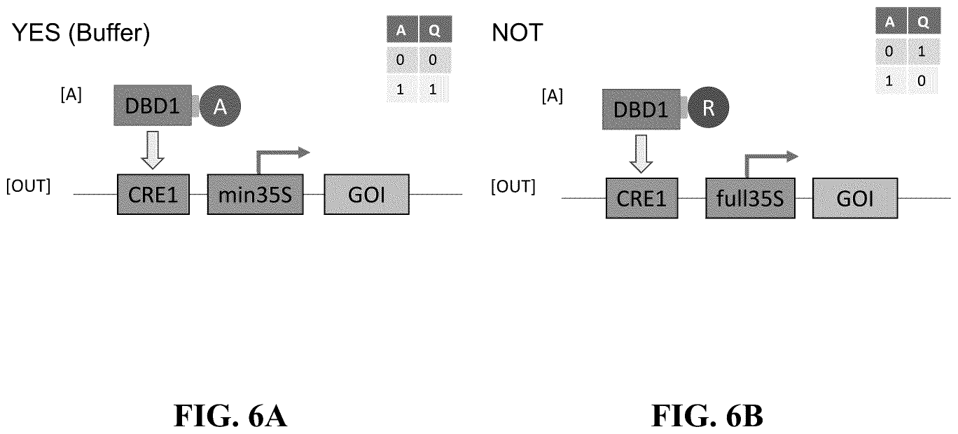

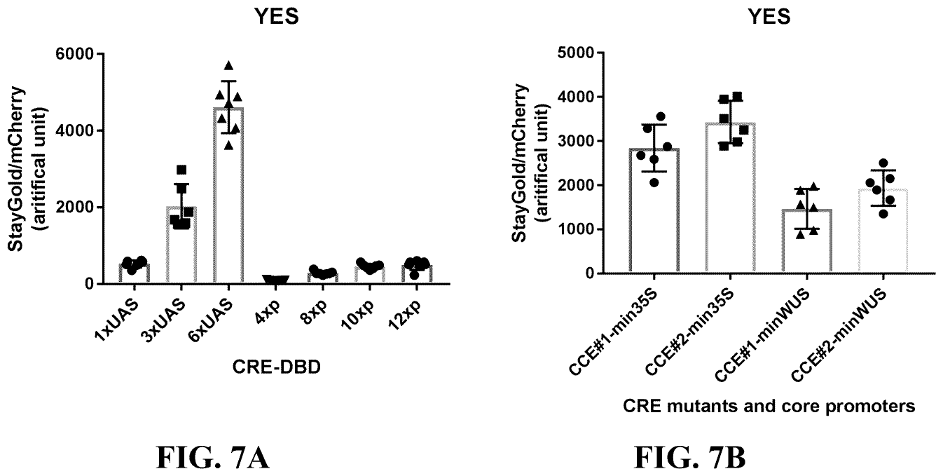

FIGS. 6A and 6B show schematics of example logic types. FIG. 6A shows a YES (Buffer) gate, which is the activation logic for OR/AND/NIMPLY/XOR gates. An activator compound binds to a DBD domain, which binds to a CRE domain, which controls a promoter (in this case min35S), which then expresses the GOI. FIG. 6B shows a NOT gate, which is the repression logic for NOR/NAND/NIMPLY/XNOR gates. A repression compound binds to a DBD, which binds to a CRE domain, which controls a promoter (in this case full35S) which may express the GOI. FIGS. 7A and 7B show the results of encoding a synthetic circuit system in tobacco leaves. A YES gate was encoded in tobacco leaves using naturally occurring CRE-DBD (FIG. 7A). A YES gate was also encoded in tobacco leaves using CRE mutants and core promoters (FIG. 7B).

TABLE 1
Exemplary DNA elements for a logic circuit.

| | Pro | 5U(f) | NT1 | Gate | Note |
|---|---|---|---|---|---|
| pL1F1#63 | En35S | 6xGal4 | -min35S-6xGal4 | NAND/IMPLY/XNOR | Single op |
| | | | | (max Repression) | |
| PL1F1#64 | En35S | 3xGal4 | -min35S-6xGal4 | NAND/IMPLY/XNOR | Single op |
| | | | | (max Repression) | |
| pL1F1#65 | En35S | 1xGal4 | -min35S-10xp43 | NOR/ | Dual op for |
| | | | | | NOR |
| pL1F1#66 | En35S | 1xGal4 | -min35S-12xp43 | NOR/ | Dual op for |
| | | | | | NOR |
| pL1F1#67 | En35S | 6xGal4 | -min35S-8xp43 | NOR/ | Dual op for |
| | | | | | NOR |
| pL1F1#68 | En35S | 3xGal4 | -min35S-8xp43 | NOR/ | Dual op for |
| | | | | | NOR |

Table 1 shows one example of the versatility of genetic logic circuits. Every DNA element in the system has many options. Specifically, the cis-regulatory element (CRE) can be DNA sequences that are targeted by Gal4 DNA-binding domain (Gal4DBD) or Zinc Finger (ZF) binding domain. Gal4DBD domain binds to DNA Gal4 upstream activation sequence (Gal4UAS). ZF43 domain binds to DNA p43. Multiple DBD-DNA pairing allows simultaneously regulating multiple output gene targets without interfering each other. The logic circuit may be 1×/3×/6×Gal4 or 4×/8×/12×p43 to increase the binding strength, which in turn increases the strength of gene activation or repression.

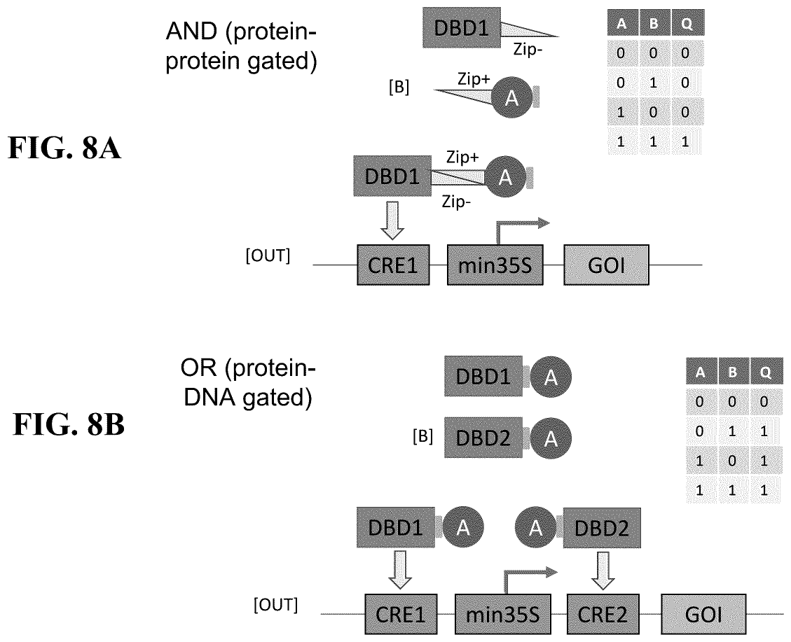

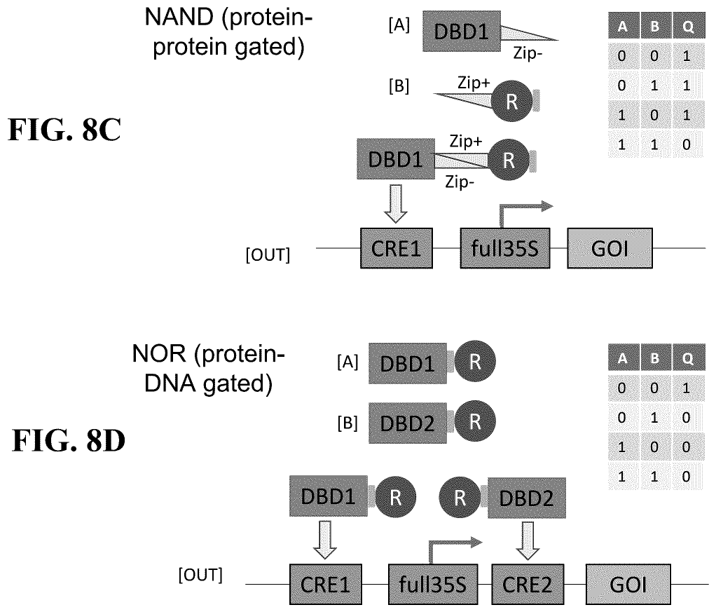

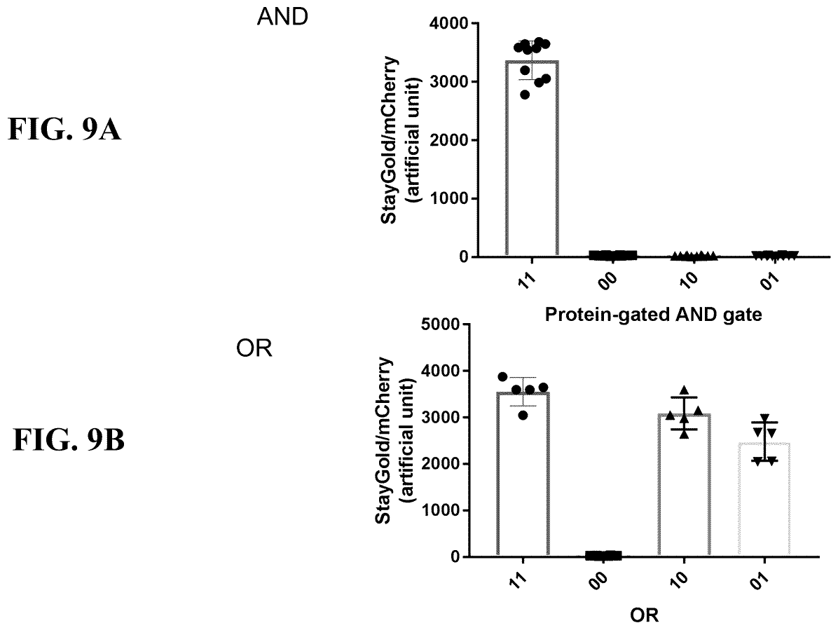

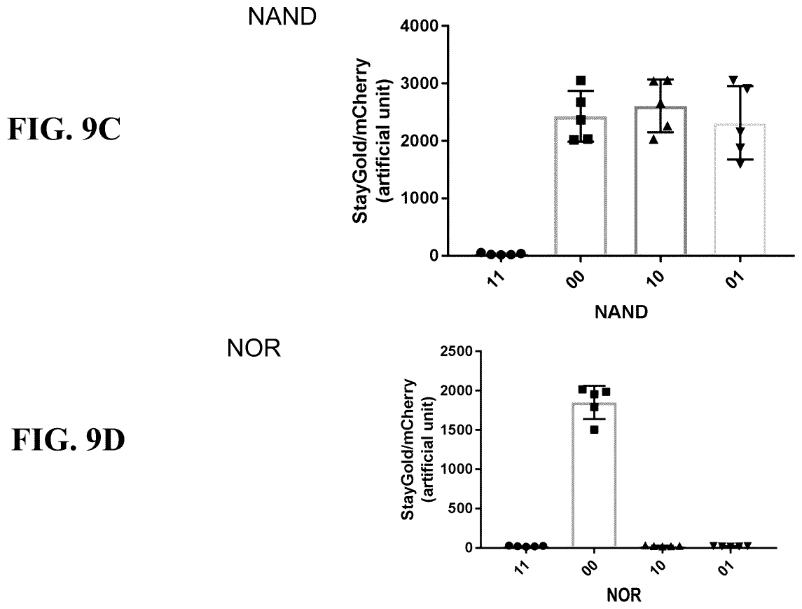

FIGS. 8A-8D show schematics of different logic gates, including AND (FIG. 8A), OR (FIG. 8B), NAND (FIG. 8C), and NOR (FIG. 8D). The AND (FIG. 8A) gate is protein-protein gated. In this gate, a single DBD domain, bound to a Zip− domain is used. The Zip+ domain is bound to an activator compound; when the two compounds bind, they then bind to the CRE domain which controls the promoter and the GOI. The OR gate (FIG. 8B) is protein-DNA gated; two DBD regions are used, referred to as DBD1 and DBD2. The same activator compound must bind to both DBD regions. Each DBD then binds a specific CRE domain, referred to as CRE1 and CRE2. The NAND gate (FIG. 8C) is protein-protein gated. A single DNA binding domain is used, and the Zip+domain binds to a repressor compound. The NOR gate (FIG. 8D) is protein-DNA gated. Two DNA binding domains are used, which bind to repressor compounds, and bind two CRE domains. FIGS. 9A-9D show the practical application of these gates in tobacco leaves. To evaluate the logic gate performance, StayGold GFP was used as an output reporter, which expression is directed by the AND/OR/NAND/NOR gate. Meanwhile, mCherry reference reporter expression is directed by a constitutive promoter pACT2. The logic gate was transformed to tobacco leaves for transient expression. For the y-axis, StayGold/mCherry fluorescence intensity ratio is an artificial unit of output. For the x-axis, 11, 00, 10, 01 represent to inputs A and B values true true, false false, true false, false true. The expression patterns match the desired expression patterns demonstrated in FIGS. 8A-8D.

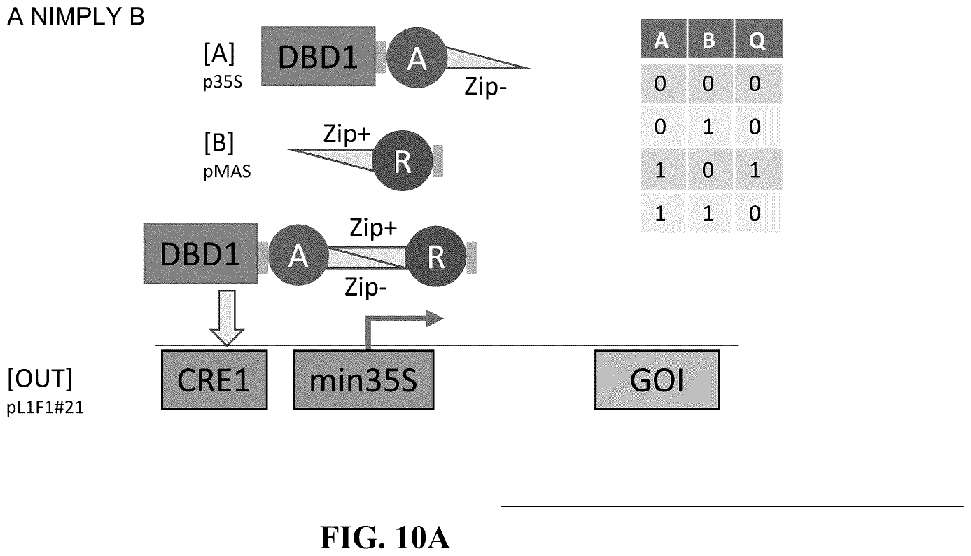

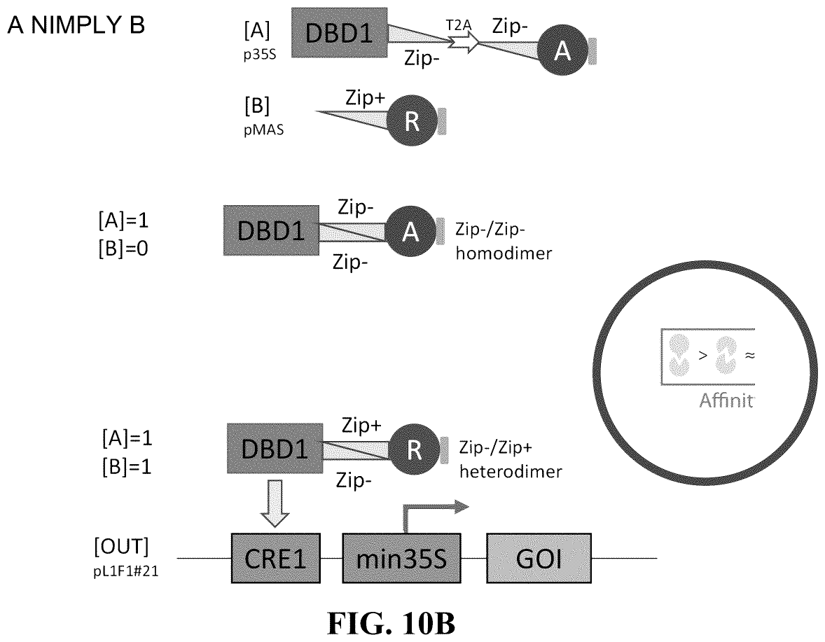

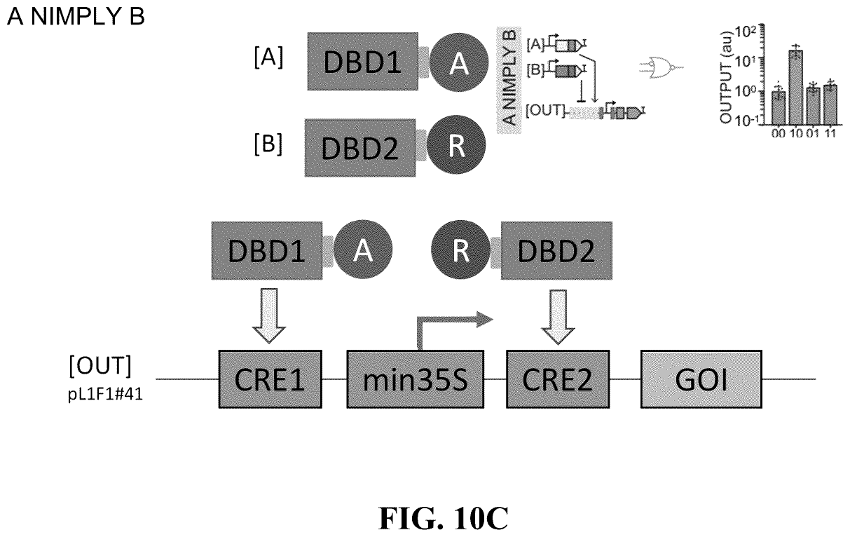

FIGS. 10A-10C show three schematics to encode a NIMPLY gate. FIG. 10A is protein-protein gated design, and uses one DBD bound to A and Zip+ bound to R. FIG. 10B is also a protein-protein gated design. Unlike FIG. 10A, T2A is bound to the Zip− domain attached to the DBD. This allows the Zip− domain to bind to another Zip− domain, forming a Zip−/Zip− homodimer. Therefore, there is competition between Zip+/R compounds and Zip−/A compounds to bind the DBD/Zip− compounds. The affinity determines which is more likely to bind and the resulting expression pattern. FIG. 10C is a protein-DNA design. In this case, two DBD are used; DBD1 binds to A, while DBD2 binds to R, each of which bind to a respective CRE domain.

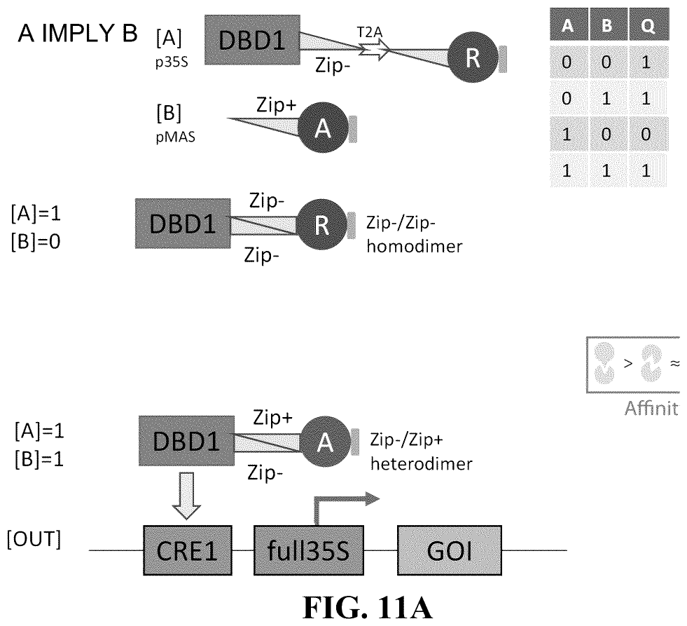

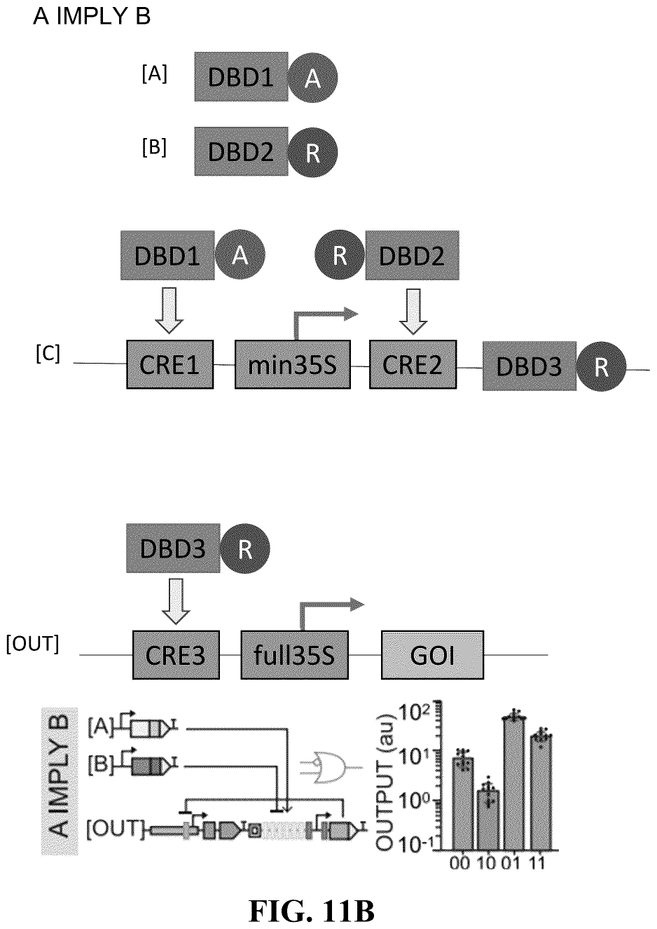

FIGS. 11A-11B show two schematics to encode an IMPLY gate. FIG. 11A is a protein-protein design. FIG. 11B is a protein-DNA gated design.

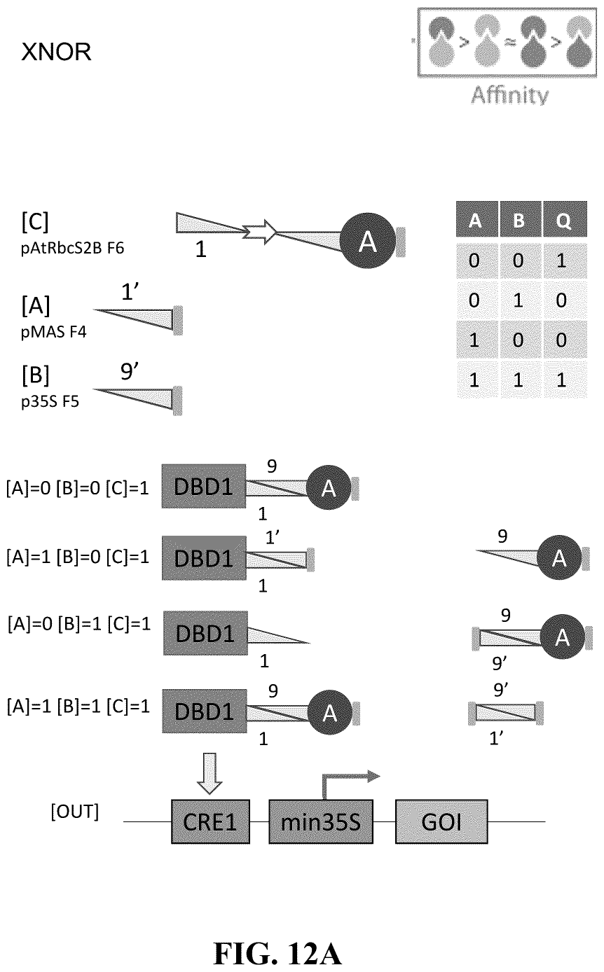

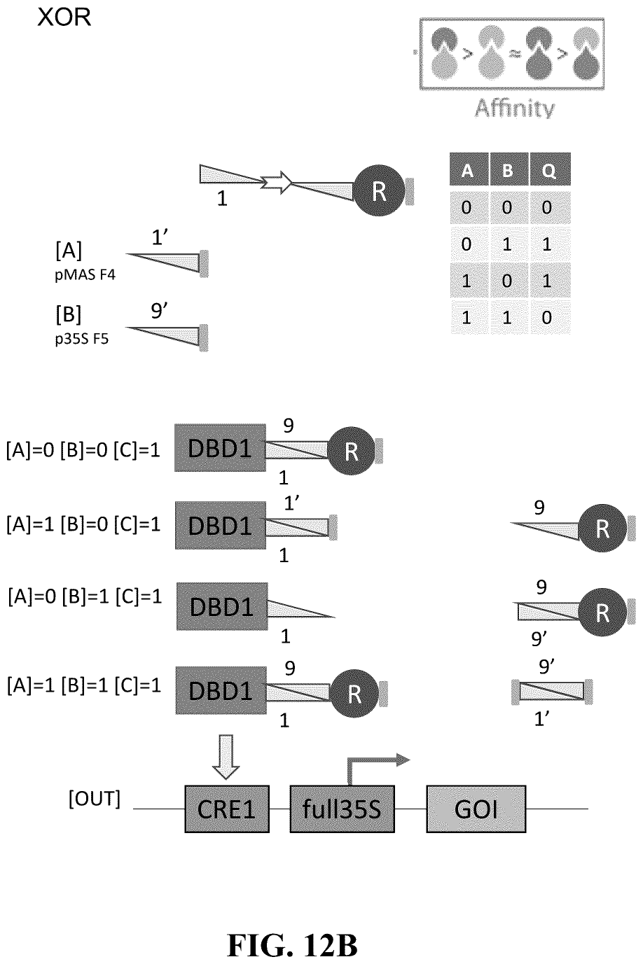

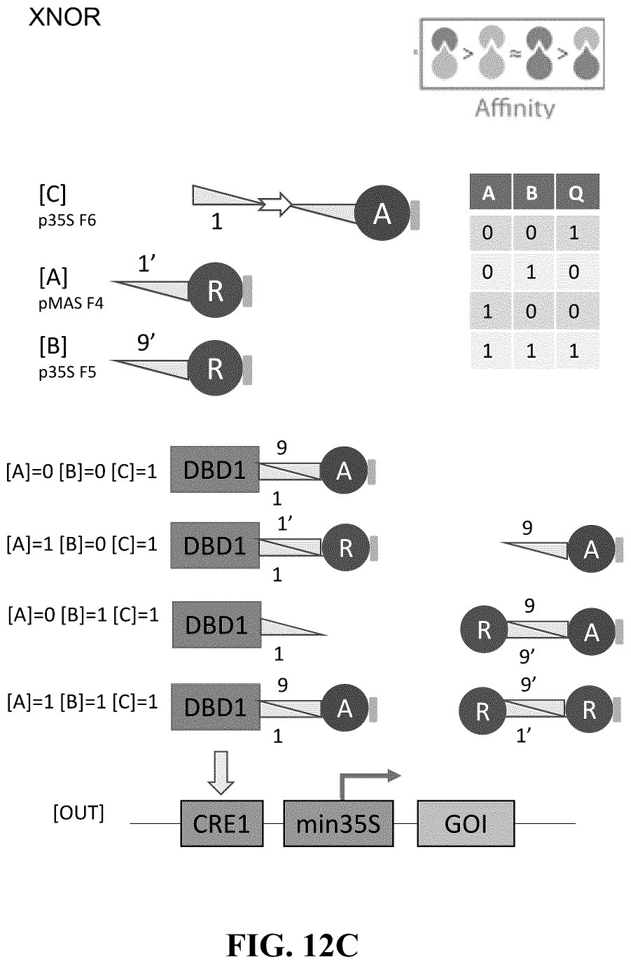

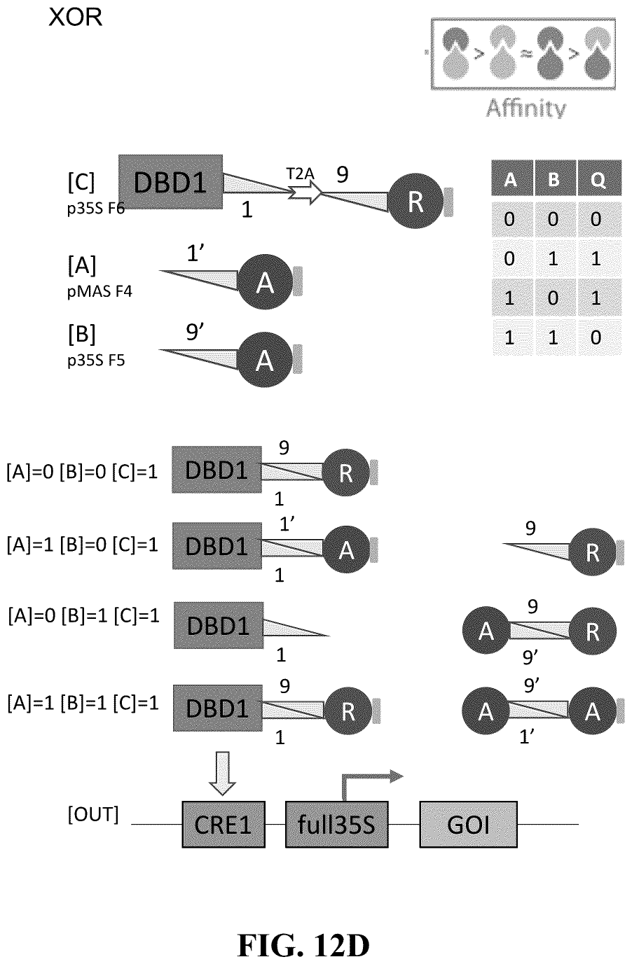

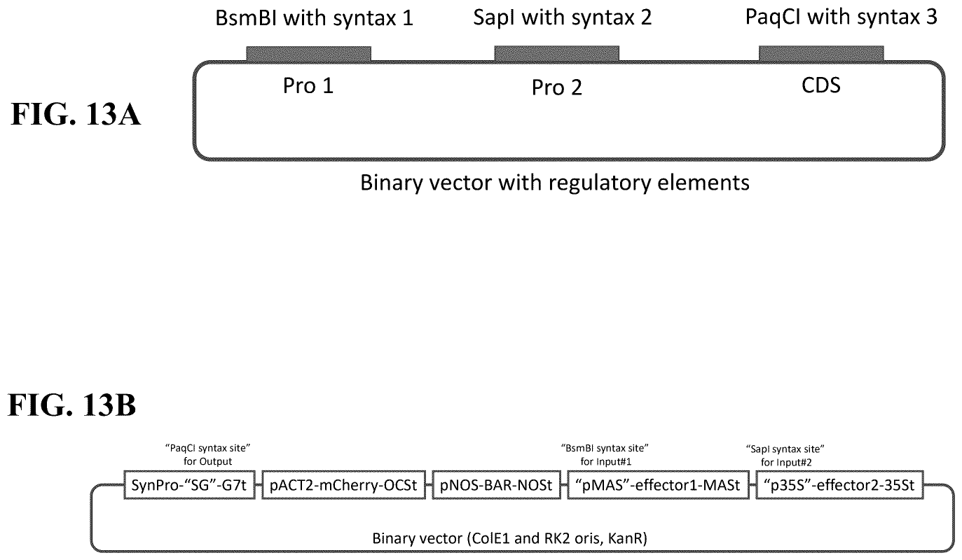

Regarding OR and exclusive XNOR gates, the fundamental issue is how to make A & B mutually destructive. FIGS. 12A-12D show schematics of designs for XNOR and XOR gates. All of the designs shown here are protein-protein gated. FIG. 12A is a XNOR gate that relies only on A (rather than A and R); FIG. 12B is a XOR gates that relies only on R. In contrast, FIGS. 12C and 12D use both A and R. FIG. 12C shows a XNOR gate and FIG. 12D shows a XOR gated.

The schematics of logic gates presented above are non-limiting. Using these basic techniques, a toolkit of customizable vectors is disclosed herein. The toolkit includes customizable binary vectors; each basic logic gate has approximately 20 customizable options. The toolkit further includes modular vectors for orthogonal protein effectors and DNA CREs; there are ~500 modular vectors in the toolkit. Over 1,100 constructs were built and tested as part of this project; every construct that performed well is included in the toolkit. The toolkit offers multiple choices for any genetic part, included DBDs, peptides, and CREs for fine-tuning and building integrated circuits.

The techniques described herein offer many benefits. High-order circuits to program the expression of multiple genes for multi-trait stacking are disclosed herein. This overcomes a critical challenge in crop breeding. Table 2 shows an overview of how this method overcomes multi-trait stacking.

TABLE 2
Example of how disclosed methods may overcome traits stacking.

| Trait A | Trait B | Trait C | Trait D | Trait X | |
|---|---|---|---|---|---|
| Root | Leaf | Drought | Oil | . . . | Logic gate |
| nematode | fungal | resistance | accumu- | | for single |
| resistance | resistance | | lation | | gene's |
| | | | | | pleiotropy |
| Gene A | Gene B | Gene C | Gene D | Gene X | |
| R gene | S gene | ABA | TF/enzyme | . . . | High order |
| | | synthesis | | | circuit for |
| | | | | | multiple |
| | | | | | traits' |
| | | | | | stacking |

Ultimate all-round competent crop

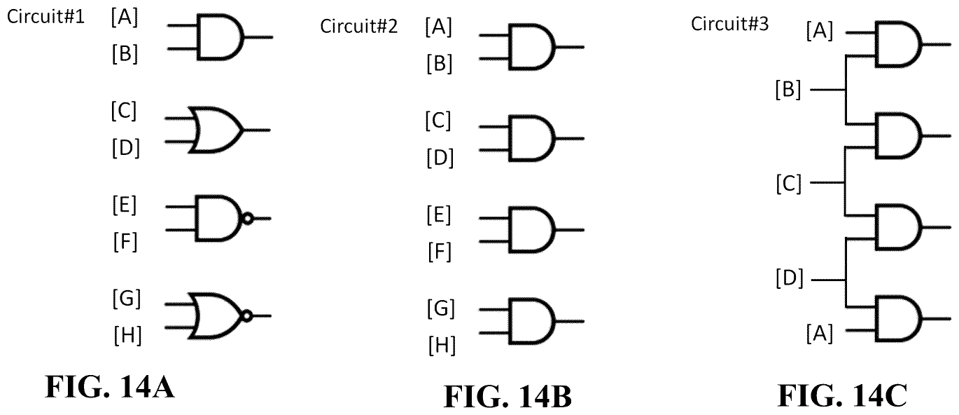

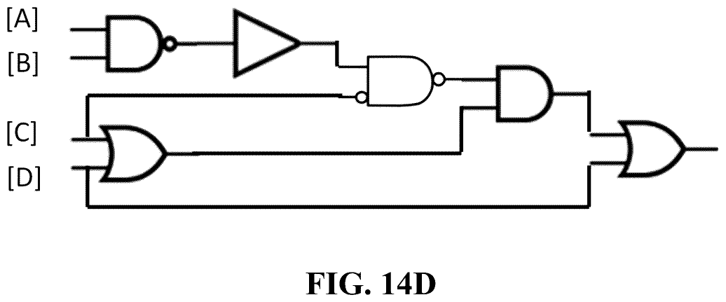

FIGS. 14A-14D show designs for integrated circuits for traits stacking. FIGS. 14A-14C (circuits 1-3) are currently part of the toolkit described above. To achieve traits stacking in plants, the inventors show stacking of a number of arbitrarily select basic logic types (FIG. 14A, AND/OR/NAND/NOR) or same logic type (FIG. 14B, four AND gates). Alternatively, a single input can be wired to different logic gates (shown in FIG. 14C), which allows dynamic up or down regulation of a target upon different inputs. FIG. 14D (circuit 4) is under development. This design circuit is a move toward programming network regulated traits (e.g., vernalization and flowering). Disclosed herein are systems and methods for building an integrated circuit that is more complex and has more layers than previously reported in other systems.

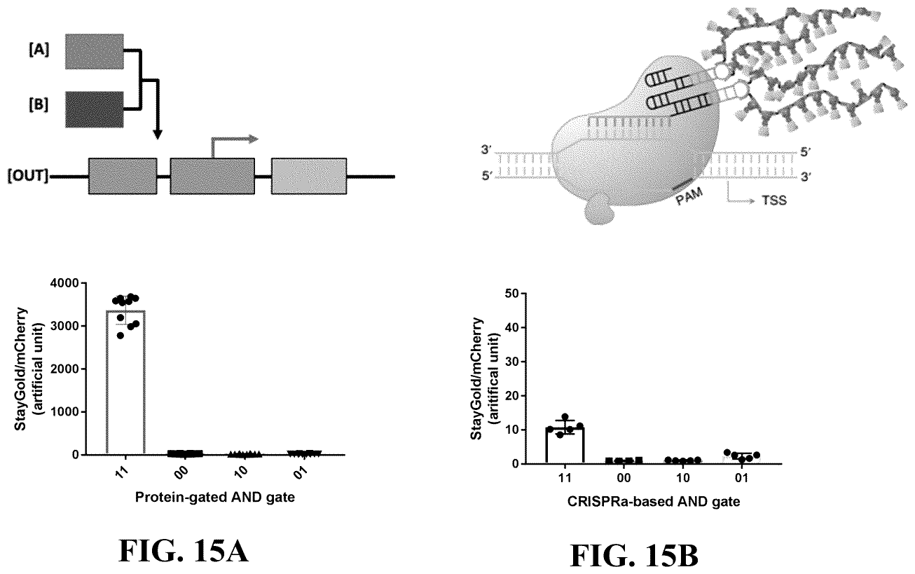

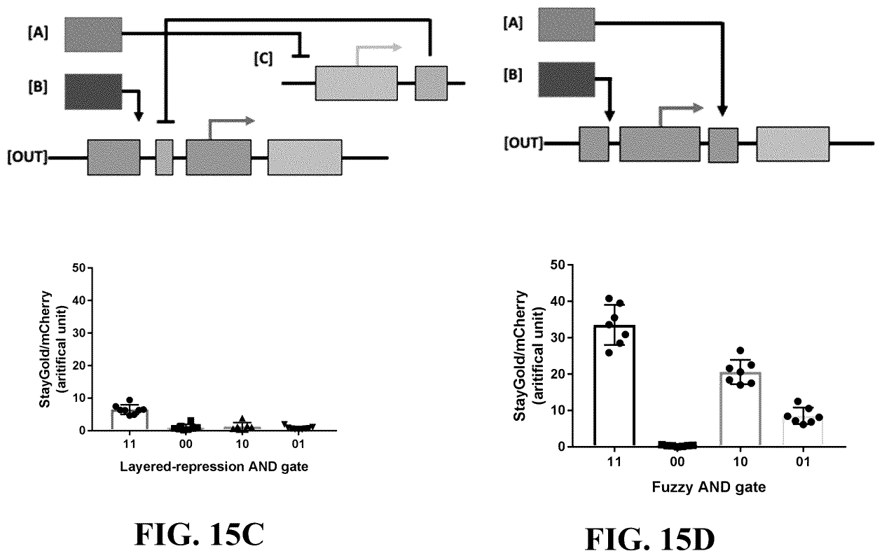

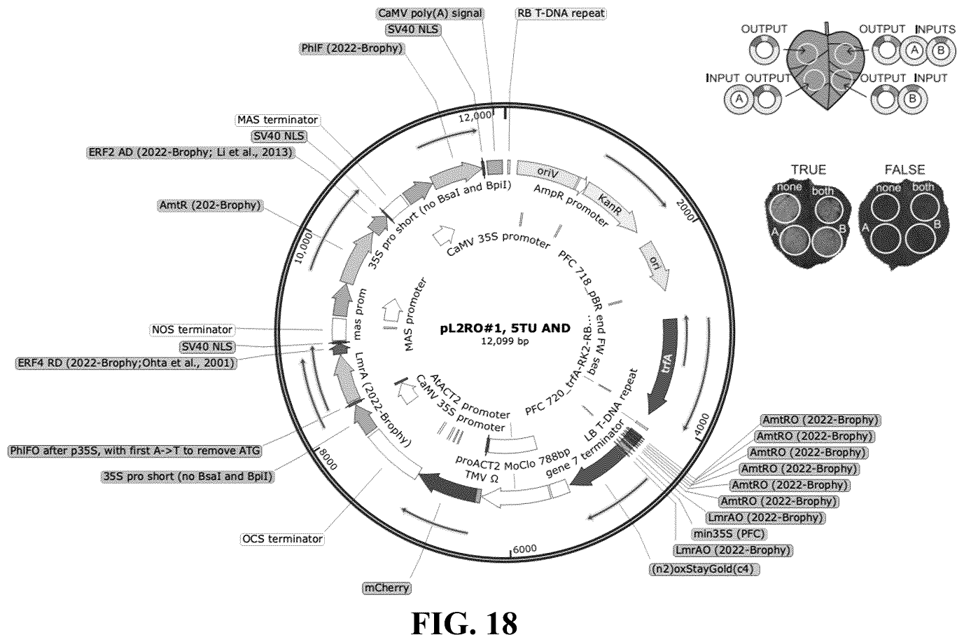

FIG. 15 shows examples for optimizing different methods of AND gate performance. FIG. 15A shows single-regulator Boolean, which is a split enhancer. FIG. 15B shows a two/three-input Boolean with split CRISPR activation. FIG. 15C shows a two-regulator Boolean with an enhancer and a repressed repressor. FIG. 18 shows an example plasmid based on this design. FIG. 15D shows a fuzzy AND gate which uses a cooperative effect.

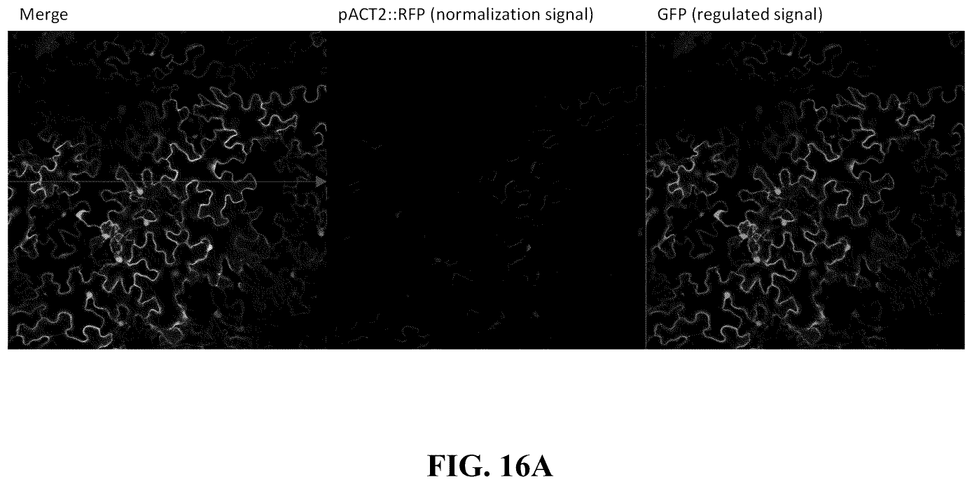

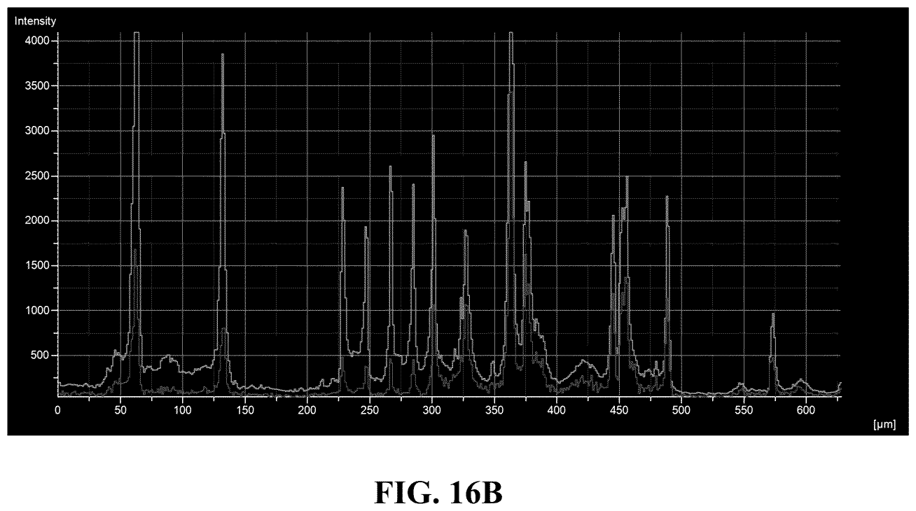

FIGS. 16A-16B show experimental results using the AND gate shown in FIG. 15A. Labeled pACT2::RFP (normalization signal) and GFP (regulated signal) were imaged and merged. A line scan was taken, and the intensity of both channels was plotted. The signals of pACT2::RFP and GFP clearly overlapped with high specificity, demonstrating that GFP was accurately regulated.

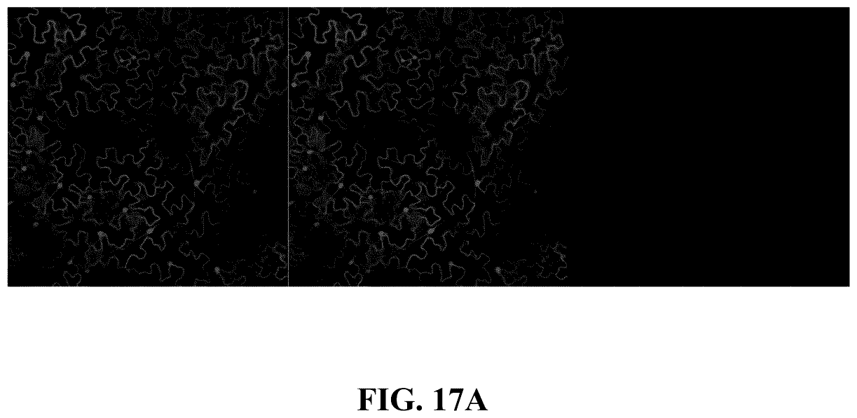

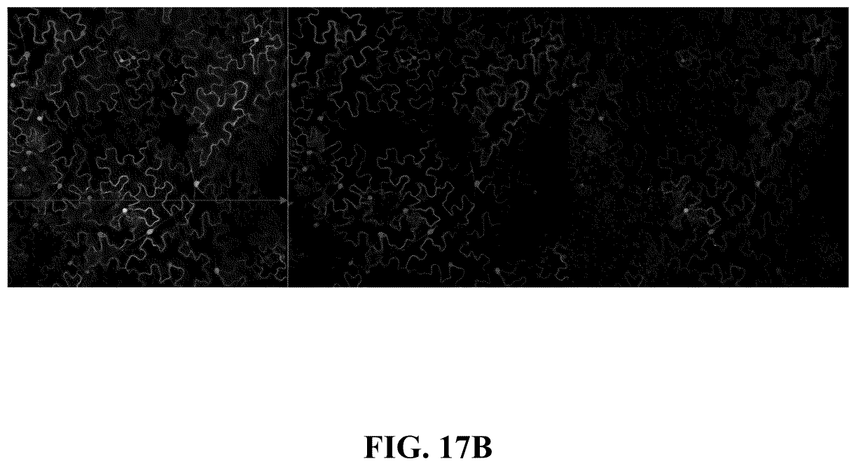

FIGS. 17A-17B show experimental results using the AND gate shown in FIG. 15A, when the AND output is designed to be false (x-axis 00). In FIG. 17A, the GFP signal is very weak, suggesting high efficiency of this AND gate regulation. In FIG. 17B, the brightness and contrast was artificially enhanced, which made the GFP signal more visible. The autofluorescence suggests that the GFP detection channel is functional and GFP expression is indeed very low.

### Example Embodiments and Implementations of Software-Based Systems and Methods

In one embodiment, systems and methods described herein may be deployed in the form of a custom circuit design service. For example, a company or other operator may perform a software-based method that receives design requirement information from a customer, and outputs a customized logic circuit and vector/introduction approach for introducing the logic circuit into a given system (or host) having an existing framework of internal input/output behavior that results in external outputs or behaviors.

Such a software-based method may allow a user to input two or more categories of initial information, including system/host information and desired/non-desired output/behavior/trait information. The system/host information may include an identification of a microorganism, plant, fungal, or animal species, or other complex host system that operates according to a prescribed set of inputs and output behaviors. For example, a plant's DNA may encode a set of growth behaviors, environmental response behaviors, desirable traits/phenotypes, undesirable traits/phenotypes, etc. Internally, the plant's DNA can be thought of as a large scale network of nodes, which may each be affected by proteins, metabolites, and other internal signals/pathways, and output various proteins, signals, etc. that affect other nodes and/or overall plant behavior and traits. Thus, the host system may have an inherent set of input/output behaviors determined according to the nature of the host system itself.

In some methods, a user may need only input information concerning a plant species or variety, such as an agricultural crop like corn, soybean, wheat, etc. The method may then consult a publicly-available (or proprietary) library of genomic information regarding that species/variety to define the inherent system behavior of the plant. In other embodiments, a user may input some or all of the genomic information regarding such plant. For example, where a customer has a proprietary plant variety for which the customer desires to introduce a genetic circuit, genomic information for such variety may not be publicly available and may need to be supplied by the customer or sequenced by the user/operator of the method.

The user-supplied output behavior information may comprise an identification of specific traits or behaviors that are desired to be exhibited (e.g., drought tolerance, higher yield, insect or disease resistance, root growth, flowering, dormancy, etc.), an identification of when/in response to what inputs those traits are desired, and/or an identification of specific traits or behaviors that are desired to be avoided (e.g., side effects of the presentation of another trait or behavior).

In further embodiments, users may also provide additional information, such as region or tissue-specific areas in which the traits or behaviors are desired/not desired, specific biomarkers that are desired to be used as “inputs”, and preferences for vectors or manner of introducing the behaviors.

With this information a system may then consult a library of information concerning the native input/outputs of the identified host/system (e.g., a library of information concerning which genes regulate/promote which behaviors and which proteins, metabolites, etc. activate those genes). In some embodiments, the system may also determine specific metabolites, biomarkers, etc. that are natively generated by the host system in response to environmental occurrences associated with the “temporal” aspects of the user's desired behavior. For example, a biomarker or other signal that is generated by a host in response to a lack of necessary resources (e.g., by a plant experience drought stress) may be utilized as a starting point by the method for designing a logic circuit. With this information, a system could utilize a path-finding algorithm (e.g., Dijkstra's algorithm, A* algorithms, etc.) to parse known genetic pathways and activities to determine an efficient way to cause expression of that signal to trigger a specific behavior (e.g., activating a gene associated with drought tolerance). In other embodiments, a logic circuit may be designed such that expression of the signal/biomarker directly activates a gene of interest.

In further embodiments, secondary systems/hosts may be utilized in combination with the system/host identified by the user, to leverage more consistent or reliable pathways for generating signals that can trigger desired behaviors. For example, various plant-associated microorganisms or fungi can be introduced to the soil in a given field or directly to a given plant/crop. That microorganism could be genetically modified to generate an output upon receipt of a given input. For example, when nitrogen levels are low, the microorganisms or fungi may express a given output (e.g., a protein, enzyme, biomarker, etc.) that is detected by the plant and utilized as an input to a synthetic logic circuit to express a given behavior (e.g., insect resistance) while depressing undesired behaviors (reduced plant growth). Thus, even for systems or hosts in which an operator lacks sufficient information to determine a signal path from a given input to a desired output, a secondary system (which is well known, reliable, and proven) may be introduced and coupled to the host to generate a signal that the host can use to exhibit the desired behavior.

Once such a system has determined the impact of the relevant inputs (and the secondary outputs they generate, such as other proteins or biomarkers within a pathway), the system can then determine a logic function that represents the desired impact of these inputs. For example, it may be that the genes regulating drought tolerance should be depressed until actual drought stress is detected. Thus, a logic circuit may accept multiple inputs in an “AND” format, including an actual biomarker indicative of drought stress, before generating an output that activates the drought tolerance genes (and/or inactivates genes that would otherwise have halted further plant growth).

This logic function can then be utilized by the system operator to lay out a series of logic gates that will implement the desired function, as described above. The system may also determine, based on the user-provided information, the appropriate implementation of this logic gate design. In some embodiments, the system may provide a circuit layout of gates as they would be developed to impact the specific genes of interest, and provide that layout to customers. In other embodiments, the system may then develop a vector that would introduce the genetic modifications of the logic gate design to a host. In other embodiments, the operator may cause the logic gate design's genetic modifications to be introduced to a given plant, and then test the degree to which these modifications reliably result in the desired outcomes and persist across generations/in seeds/etc. Then, germplasm or seeds that exhibit the logic gate's behavior in a stable way can be provided to the customer.

What is claimed is:

1. A method for generating an application-specific, modular circuit vector to implement a set of logic functions comprising:
receiving user input comprising data indicative of: a crop variety of interest, one or more desired traits, one or more undesired traits, and one or more genes of interest;
accessing a library of gene information pertaining to the crop variety of interest, to obtain gene activation information relating to the one or more desired traits, one or more undesired traits, and one or more genes of interest;
using the gene activation information, generating a circuit layout comprising a plurality of logic gates wherein an input channel to the circuit layout corresponds to an input that affects the one or more genes of interest to affect the one or more desired traits and the one or more undesired traits;
compiling a modular vector to encode the logic gates in the circuit layout for the crop variety of interest by:
identifying a stored vector construct, from a database of vector constructs, for each class of logic gate used in the circuit layout;
customizing the vector constructs to encode at least one logic gate input for each vector construct, the logic gate input associated with either: inputs identified as part of the gene activation information, or logic gate outputs of other vector constructs within the circuit layout;
customizing at least one of the vector constructs to encode a logic gate output associated with the one or more genes of interest;
combining the customized vector constructs to form the modular vector expressing the designed circuit layout; and
outputting a file to the user comprising sequence information of a DNA vector corresponding to the modular vector.

2. The method of claim 1, wherein the input channel to the circuit layout is driven by the presence or absence of the input, the input comprising at least one of: a biomarker indicative of a disease state; a biomarker indicative of a stress state; or a protein that regulates the one or more genes of interest.

3. The method of claim 1, wherein the circuit layout further comprises at least one output channel corresponding to the logic gate output and expressing an output that comprises an ‘on’ or ‘off’ regulation of one or more genes causing promotion of the one or more desired traits with depression of the one or more undesired traits.

4. The method of claim 1, wherein the database of vector constructs comprises stored vector constructs for each of the following classes of logic ages: NOT, AND, NAND, OR, NOR, XOR, XNOR, IMPLY, and NIMPLY.

5. The method of claim 4, wherein the vector constructs for each of the classes of logic gates use a common mechanism and are configured to modularly connect to one another for consistent, serial operation.

6. The method of claim 5, wherein the mechanism is DNA operon-based transcriptional regulation.

7. The method of claim 5, wherein the mechanism is RNA operon-based transcriptional regulation.

8. The method of claim 1, wherein the modular vector comprises at least three vector constructs linked in series, wherein the logic gate output of two of the at least three vector constructs are inputs to logic gate inputs of other vectors.

9. The method of claim 1, further comprising formulating a logic function based on the activation information relating to the one or more desired traits, one or more undesired traits, and one or more genes of interest, the logic function defining:
first inputs promoting gene activity of a first plurality of genes that up-regulate the one or more desired traits;
second inputs promoting gene activity of a second plurality of genes that down-regulate the one or more undesired traits; and
at least one conditional function that: (i) causes activation, through at least one second input, of at least one gene of the second plurality of genes that down-regulates the one or more undesired traits, while (ii) causing activation, through at least one first input, of at least one gene of the first plurality of genes that up-regulates the one or more desired traits but also corresponds with up-regulation of the one or more undesired traits.

10. The method of claim 1 further comprising transfecting a sample of the crop of interest to contain the modular vector.

11. A circuit comprising:
one or more input channels configured to sense one or more inputs;
a plurality of logic gates arranged in a circuit layout having at least three layers, each logic gate having at least one logic gate input and at least one logic gate output;
one or more output channels configured to activate a gene of interest;
wherein the logic gate input for each of the plurality of logic gates comprises at least one of: a DNA binding domain bound to a zip domain, a zip domain bound to an activation domain; or a zip domain bound to a repression domain; and
wherein the circuit layout implements a logic function comprising: a first gene of a crop variety to be activated upon sensing of the one or more inputs, and a second gene of the crop variety to be repressed in association with activation of the first gene.

12. The circuit of claim 11, wherein the one or more inputs comprise at least one of: a disease biomarker or an environmentally-caused biological signal.

13. The circuit of claim 11, wherein the first gene comprises a tissue-specific promotor and corresponds to a desired trait.

14. The circuit of claim 13, wherein the second gene corresponds to an undesired trait that, but for the circuit layout, would be activated in association with activation of the first gene.
